(19) United States
(12) Patent Application Publication
(10) Pub. No.: US 2023/0143377 A1
(43) Pub. Date: May 11, 2023

(54) GAS CHROMATOGRAPH FOR UNDERGROUND MINE

(71) Applicant: CCTEG Shenyang Research Institute, Fushun (CN)

(72) Inventors: Yuntao Liang, Fushun (CN); Junjie Zhang, Fushun (CN); Yong Sun, Fushun (CN); Xuewei Ge, Fushun (CN); Wenjie Fang, Fushun (CN); Jie Ren, Fushun (CN); Depeng Zhang, Fushun (CN); Cheng'ao Fu, Fushun (CN); Jilin Cui, Fushun (CN); Honghao He, Fushun (CN); Dong Zhang, Fushun (CN)

(21) Appl. No.: 17/522,859

(22) Filed: Nov. 9, 2021

(51) Int. Cl. G01N 30/20; G01N 30/60; G01N 30/66

(52) U.S. Cl. CPC G01N 30/20; G01N 30/66; G01N 30/6052; G01N 2030/025

(57) ABSTRACT

The disclosure includes a gas chromatograph for underground use comprising a fixing board. In some embodiments, the gas chromatograph for underground use includes a power connection socket, at least one three-way solenoid valve, a quantitative loop, an analysis module, a communication main board, a data transmission device, a sample injection membrane valve, and a gas injection pump detachably coupled to the fixing board. In some embodiments, the quantitative loop and the sample injection membrane valve are arranged and configured to couple to at least one of the at least one three-way solenoid valves.

### BACKGROUND

### Field

The invention relates to mine gas analysis technology. In particular, the invention relates to a gas chromatograph for underground mining use.

### Description of Related Art

Gas chromatography as a technological field has been developing for over 50 years and has become a widely used analytical technology for separating complex mixtures. Gas chromatography is extensively applied to petrochemical analysis, drug analysis, food analysis, environmental analysis, and polymer analysis. Gas chromatography is essential in industrial applications, agriculture, national defense, construction, and scientific research.

Gas chromatographs analyze and detect various components in mixed gases by using a carrier gas system to take the gas sample into the chromatographic column for separation. A detector is responsible for the detection of the composition of the gas sample.

Gas chromatographs are used in various fields, including the petrochemical industry, medicine and health, and the food industry, among others. This is because gas chromatographs can be used for quantitative and qualitative analysis and to determine physicochemical constants such as the distribution coefficient and activity coefficient of a sample during a stationary phase.

The prior art has several limitations, which will be described in greater detail. Thus, there is a need for systems and methods that remedy the many prior art deficiencies.

### SUMMARY

The disclosure includes a gas chromatograph for underground mine use comprising a fixing board. In some embodiments, the gas chromatograph for underground mine use includes a power connection socket detachably coupled to the fixing board. According to some embodiments, the gas chromatograph for underground mine use comprises at least one three-way solenoid valve detachably coupled to the fixing board. The gas chromatograph for underground mine use may comprise a quantitative loop detachably coupled to the fixing board, the quantitative loop arranged and configured to detachably couple to at least one of the at least one three-way solenoid valves. In some embodiments, the gas chromatograph for underground mine use comprises an analysis module detachably coupled to the fixing board. According to some embodiments, the gas chromatograph for underground mine use comprises a communication main board detachably coupled to the fixing board. The gas chromatograph for underground mine use may include a data transmission device detachably coupled to the fixing board. In some embodiments, the gas chromatograph for underground mine use comprises a sample injection membrane valve detachably coupled to the fixing board, the sample injection membrane valve arranged and configured to detachably couple to at least one of the at least one three-way solenoid valves. According to some embodiments, the gas chromatograph for underground mine use comprises a gas injection pump detachably coupled to the fixing board.

The analysis module, the sample injection membrane valve, the gas injection pump, the quantitative loop, the at least one three-way solenoid valve, the data transmission device, the communication main board, and the power connection socket may be detachably coupled to the fixing board with clips and screws.

In some embodiments, the gas chromatograph for underground mine use further comprises a housing. The fixing board may be detachably coupled to a fixed column at a bottom of the housing with screws.

According to some embodiments, the data transmission device further comprises parallel RJ45 and RS485 transmission interfaces arranged and configured to couple to the communication main board.

The gas chromatograph for underground mine use may further comprise a sample gas injection port arranged and configured to detachably couple to at least one of the at least one three-way solenoid valves. In some embodiments, the gas chromatograph for underground mine use comprises a sample gas exhaust port arranged and configured to allow a sample gas to flow out of the sample gas injection port. According to some embodiments, the gas chromatograph for underground mine use comprises a standard gas injection port arranged and configured to detachably couple to at least one of the at least one three-way solenoid valves. The gas chromatograph for underground mine use may comprise a gas injection pump exhaust port arranged and configured to allow a standard gas to flow out of the standard gas injection port. In some embodiments, the gas chromatograph for underground mine use comprises an analysis module exhaust port arranged and configured to allow the sample gas and the standard gas to flow out of the analysis module. According to some embodiments, the gas chromatograph for underground mine use comprises a carrier gas interface arranged and configured to detachably couple to the analysis module and drive the sample gas and the standard gas into the analysis module through the use of carrier gas.

The analysis module may include a capillary chromatographic column equipped with a heating module. In some embodiments, the analysis module comprises a micro TCD detector equipped with a heating module.

According to some embodiments, the gas chromatograph for underground mine use comprises a housing, wherein the carrier gas interface, the gas injection pump exhaust port, the sample gas injection port, the standard gas injection port, the analysis module exhaust port, and the sample gas exhaust port are detachably coupled to the housing. The carrier gas interface, the gas injection pump exhaust port, the sample gas injection port, the standard gas injection port, the analysis module exhaust port, and the sample gas exhaust port may further comprise a fixed nut and a joint body, the joint body filled in with powder metallurgy of particle size 60 to 80 mesh.

In some embodiments, at least one three-way solenoid valve further comprises a first three-way solenoid valve and a second three-way solenoid valve. According to some embodiments, the first three-way solenoid valve further comprises an A end coupled to the sample injection membrane valve. The first three-way solenoid valve may include a P end coupled to the standard gas injection port. In some embodiments, the first three-way solenoid valve comprises an R end coupled to the second three-way solenoid valve.

According to some embodiments, the second three-way solenoid valve further comprises an A end coupled to the quantitative loop. The second three-way solenoid valve may further comprise a P end coupled to the sample injection gas port. In some embodiments, the second three-way solenoid valve further comprises an R end coupled to the first three-way solenoid valve.

According to some embodiments, the sample injection membrane valve comprises a four-way valve coupled to a suction end of the gas injection pump, the quantitative loop, the carrier gas interface, and the analysis module. A sample inlet end of the sample membrane valve may be coupled to the suction end of the gas injection pump when the sample membrane valve is not activated. In some embodiments, the sample membrane valve’s carrier gas interface end is coupled to the carrier gas interface. An analysis module connecting the end of the sample membrane valve may be coupled to the analysis module when the sample membrane valve is activated.

The disclosure also includes a method of using a gas chromatograph wherein the gas chromatograph comprises a first three-way solenoid valve including an A end and a P end, a second three-way solenoid valve including an A end and a P end, a standard gas injection port, a sample injection membrane valve, a gas injection pump, a standard gas, a carrier gas, at least one sample gas, an analysis module, a quantitative loop, and a sample gas injection port. According to some embodiments, the method of using a gas chromatograph comprises calibrating the gas chromatograph. The method of using a gas chromatograph may include analyzing the at least one sample gas.

In some embodiments, calibrating the gas chromatograph further comprises powering on the first three-way solenoid valve. According to some embodiments, calibrating the gas chromatograph includes coupling, via the A end and the P end of the first three-way solenoid valve, the standard gas injection port, the sample injection membrane valve, and the gas injection pump. Calibrating the gas chromatograph may further comprise injecting the standard gas into the sample injection membrane via the gas injection pump. In some embodiments, calibrating the gas chromatograph comprises activating the sample injection membrane valve. According to some embodiments, calibrating the gas chromatograph comprises allowing, via activating the sample injection membrane valve, at least one of the at least one sample gas into the analysis module.

Analyzing the at least one sample gas may further comprise coupling, via the A end and the P end of the second three-way solenoid valve, the second three-way solenoid valve, the quantitative loop, and the sample gas injection port. In some embodiments, analyzing the at least one sample gas comprises sending, via the coupling of the second three-way solenoid valve, the quantitative loop, and the sample gas injection port, at least one sample gas through the sample gas injection port and into the quantitative loop. According to some embodiments, analyzing the at least one sample gas comprises injecting, via the gas injection pump, the at least one sample gas into the sample injection membrane valve from the quantitative loop. Analyzing the at least one sample gas may comprise activating the sample injection membrane valve. In some embodiments, analyzing the at least one sample gas comprises allowing, via activating the sample injection membrane valve, the carrier gas to drive the at least one sample gas into the analysis module; and testing the at least one sample gas for analysis.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIG. 1 illustrates a diagrammatic view of internal connections of the gas chromatograph, according to some embodiments.

FIG. 2 illustrates a diagrammatic view of the gas interface, according to some embodiments.

FIG. 3 illustrates a flowchart depicting a method of using a gas chromatograph, according to some embodiments.

FIG. 4 illustrates a flowchart depicting a method of calibrating a gas chromatograph, according to some embodiments.

FIG. 5 illustrates a flowchart depicting a method of analyzing at least one sample gas, according to some embodiments.

### DETAILED DESCRIPTION

Although specific embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order-dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### COMPONENT INDEX

1 - Uninterruptable power supply in the underground coal mine
2 - Gas chromatograph for underground mine use
3 - Network switch
4 - Computer
20 - Fixing board
21 - Power connection socket
22 - First three-way solenoid valve
23 - Second three-way solenoid valve
24 - Sample gas injection port
25 - Quantitative loop
26 - Sample gas exhaust port
27 - Standard gas injection port
28 - Gas injection pump exhaust port
29 - Carrier gas interface
211 - Analysis module
212 - Analysis module exhaust port
213 - Communication main board
214 - Data transmission device
215 - Sample injection membrane valve
216 - Gas injection pump
241 - Copper pipe joint connection port
242 - Movable nut
243 - Joint body
244 - Powder metallurgy
245 - Fixed nut
246 - Hose joint connection port

The gas chromatograph for underground mine use is mainly applied to coal mines for analyzing concentrations of harmful gases underground to ensure safe production within the mines. Currently, gas chromatographs for mine use are primarily used at ground level - not within the mine. The gases are sampled manually and then delivered to the ground monitoring room for analysis. This wastes a great deal of manpower and material resources for the sample delivery. The analysis is also very time-consuming and fails to perform the analysis in real-time.

The underground beam tube monitoring system disclosed in the patent “Vehicle-mounted, ground and underground beam tube emergency rescue command system” (Patent No. ZL 2011 2 0356352.2), applied for by CCTEG Shenyang Research Institute, combined all equipment for a gas chromatograph and designed an overall encompassing housing for general mine use. However, no changes were made to the design structure of the gas chromatograph itself. Furthermore, the system could only be applied to the main air inlet roadway of low gas mines, thus limiting potential locations suitable for the application of the invention.

In application, a gas chromatograph for use in a mine needs to be calibrated before conducting underground gas analysis. When the standard gas and the sample gas are injected at ground level, the pressure valve of the external standard gas cylinder needs to be manually screwed to release the standard gas into the sample bag. During this process, potential standard gas leakage will not affect production in the mine. However, when a mine gas chromatograph is used underground, safe production in the mine will be affected when standard gas leakage occurs. Additionally, due to the conditions within a mine, such as an underground coal mine, it is beneficial to have fewer underground workers as a safety precaution.

Therefore, it would be beneficial to automate underground gas chromatographs. The provided invention for a gas chromatograph for underground mine use may be used for daily monitoring and emergency rescue efforts within mines. These use case scenarios are facilitated by shortening the gas sampling time and improving the real-time performance of the analysis.

The disclosure herein improves the level of automation for gas chromatographs for mine use. The disclosure also enables gas chromatographs to be used underground by optimizing the structure itself and the housing design. Benefits of this disclosure include improvements in the real-time performance and the speed of gas analysis, which is conducive to improvements in overall mine safety and the timing and speed at which accidents may be handled.

### Mode of Fabricating the Chromatograph

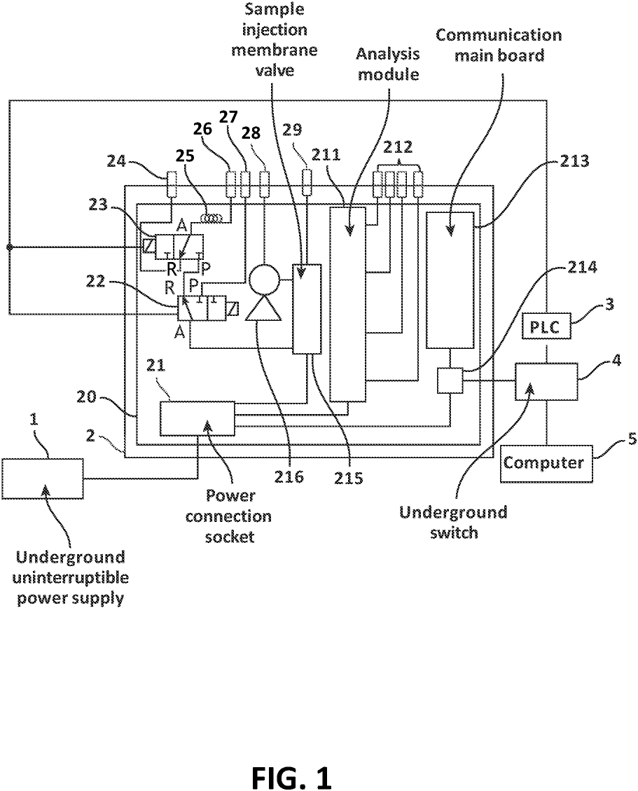

As illustrated in FIG. 1, a gas chromatograph for underground mine use 2 may comprise a fixing board 20, a power connection socket 21, a first three-way solenoid valve 22, a second three-way solenoid valve 23, a sample gas injection port 24, a quantitative loop 25, a sample gas exhaust port 26, a standard gas injection port 27, a gas injection pump exhaust port 28, and a carrier gas interface 29. In some embodiments, the gas chromatograph for underground mine use 2 includes an analysis module 211, an analysis module exhaust port 212, a communication main board 213, a data transmission device 214, a sample injection membrane valve 215, and a gas injection pump 216.

According to some embodiments, the sample gas injection port 24, the sample gas exhaust port 26, the standard gas injection port 27, the gas injection pump exhaust port 28, the carrier gas interface 29, and the analysis module exhaust port 212 are coupled to the housing. The movable nut 242 may be coupled to the inside of the housing, and the fixed nut 245 may be coupled to the outside of the housing.

In some embodiments, the analysis module 211, the communication main board 213, the sample injection membrane valve 215, and the data transmission device 214 are detachably coupled with clips and then detachably coupled to the fixing board with screws.

Some embodiments include the first three-way solenoid valve 22, the second three-way solenoid valve 23, the gas injection pump 216, the sample injection membrane valve 215, the quantitative loop 25, and the power connection socket 21 are coupled to the fixing board 20 by nuts. The fixing board 20 may be coupled to a fixed column at the bottom of the housing with screws.

In some embodiments, the first three-way solenoid valve 22 and the second three-way solenoid valve 23 include A, R, and P ends. According to some embodiments, the A end of the first three-way solenoid valve 22 is coupled to the injection port of the sample injection membrane valve 215. The R end of the first three-way solenoid valve 22 may couple to the P end of the second three-way solenoid valve 23. In some embodiments, the P end of the first three-way solenoid valve 22 couples to the standard gas injection port 27 through a stainless-steel tube. According to some embodiments, the R end of the second three-way solenoid valve couples to the sample gas injection port 24 through a soft copper tube. The A end of the second three-way solenoid valve may directly couple to the quantitative loop 25.

In some embodiments, the suction port of the gas injection pump 216 couples to the sample injection membrane valve through a stainless-steel tube. According to some embodiments, the exhaust port of the gas injection pump 216 couples to the gas injection pump exhaust port 28 through a plastic tube.

Eight power lines may electrically couple to the power connection socket. According to some embodiments, these eight power lines include electrical coupling for the first three-way solenoid valve 22, the second three-way solenoid valve 23, the analysis module 211, the communication main board 213, the data transmission device 214, the sample injection membrane valve 215, and the gas injection pump 216.

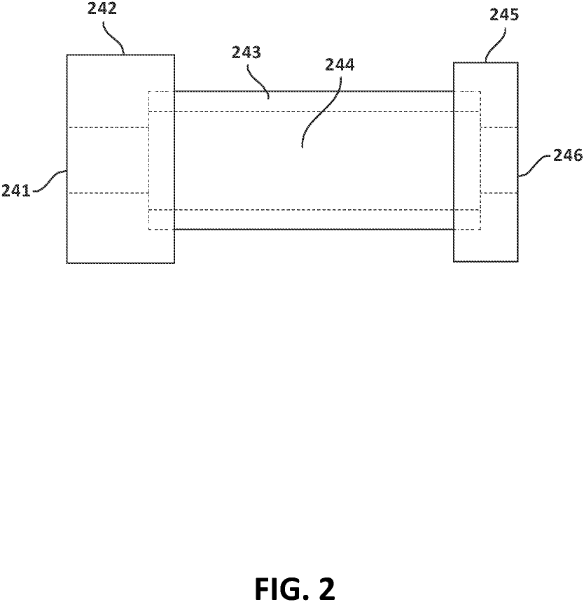

As illustrated in FIG. 2, in the gas interface device, the fixed nut 245 may be integrated with the joint body 243. In some embodiments, a hose joint is installed at the hose joint connection port 246 of the fixed nut 245. According to some embodiments, a copper tube joint is installed at the copper tube joint connection port on the side of the movable nut 242, and the movable nut 242 is tightly coupled to the joint body 243. The joint body 243 may be filled in by powder metallurgy of particle size 60 to 80 mesh.

### Procedure of Using the Chromatograph

As illustrated in FIG. 1, during the chromatograph operation in an underground coal mine, stable uninterrupted power may be provided for the device by the uninterruptible power supply in the unground coal mine 1. According to some embodiments, the first three-way solenoid valve 22 and the second three-way solenoid valve 23 are controlled by a PLC.

During calibration of the gas chromatograph, a calibration option may be selected through the software on the computer 4. According to some embodiments, first, the first three-way solenoid valve 22 is powered on, and couple the sample injection membrane valve 215 to the standard gas injection port 27 through the A end and the P end of the first three-way solenoid valve. In some embodiments, the next step is using the gas injection pump 216 to send the standard gas in the external standard gas cylinder to the sample injection membrane valve 215 for testing. After this, the sample injection membrane valve 215 may be powered on.

In some embodiments, the carrier gas drives the standard gas into the analysis module 211 through the carrier gas interface 29, where a micro TCD then detects the standard gas after being separated by a capillary chromatographic column in the analysis module. According to some embodiments, the output electrical signal is uploaded to computer 4 via the data transmission device 214 by the underground switch 3. The calibration may then be conducted by computer 4 according to a standard gas value input entered into the computer 4 ahead of time.

In some embodiments, after calibration is completed, the standard gas is inspected until the analysis results meet the quantitative repeatability inspection standard specified in “GBT 30431-2013 Laboratory Gas Chromatograph.” According to some embodiments, the procedure finishes with analyzing the underground gases.

In some embodiments, when the underground gases are being analyzed, the first step is to use the external sample gas injection device to inject the sample gas into the quantitative loop 25 via the sample gas injection port 24 and the R end of the second three-way solenoid valve 23. According to some embodiments, the next step is to power on the second three-way solenoid valve 23 and couple the quantitative loop 25 to the R end of the first three-way solenoid valve 22 through the A end and the P end of the second three-way solenoid valve 23. After this, the gas injection pump 216 may inject gases from the quantitative loop 25 into the sample injection membrane valve 215 for testing.

In some embodiments, the next step is to power on the sample injection membrane valve 215. According to some embodiments, the carrier gas drives the standard gas into the analysis module 211 through the carrier gas interface 29. The micro TCD may then detect the standard gas after being separated by a capillary chromatographic column in the analysis module. In some embodiments, the output electrical signal is uploaded to computer 4 via the data transmission device 214 by the underground switch 3. According to some embodiments, at the end of the analysis, the analysis result is output on computer 4.

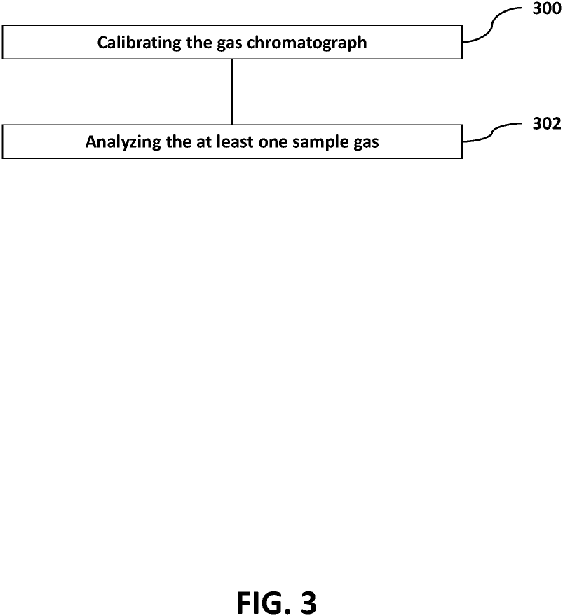

FIG. 3 illustrates a method of using a gas chromatograph, wherein the gas chromatograph comprises a first three-way solenoid valve including an A end and a P end, a second three-way solenoid valve including an A end and a P end, a standard gas injection port, a sample injection membrane valve, a gas injection pump, a standard gas, a carrier gas, at least one sample gas, an analysis module, a quantitative loop, and a sample gas injection port. In some embodiments, the method includes calibrating the gas chromatograph (at step 300). According to some embodiments, the method includes analyzing the at least one sample gas (at step 302).

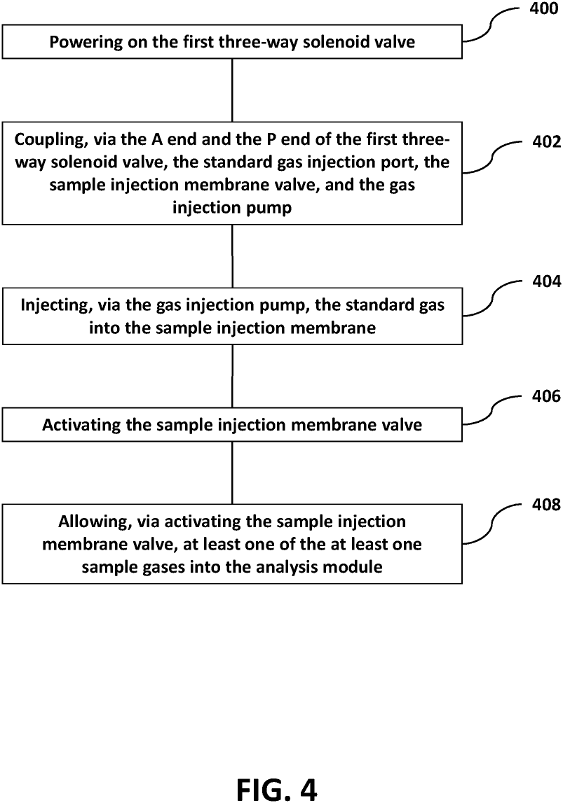

FIG. 4 illustrates a method of calibrating a gas chromatograph. The method may include powering on the first three-way solenoid valve (at step 400). In some embodiments, the method includes coupling, via the A end and the P end of the first three-way solenoid valve, the standard gas injection port, the sample injection membrane valve, and the gas injection pump (at step 402). According to some embodiments, the method includes injecting, via the gas injection pump, the standard gas into the sample injection membrane (at step 404). The method may include activating the sample injection membrane valve (at step 406). In some embodiments, the method comprises allowing, via activating the sample injection membrane valve, at least one of the at least one sample gases into the analysis module (at step 408).

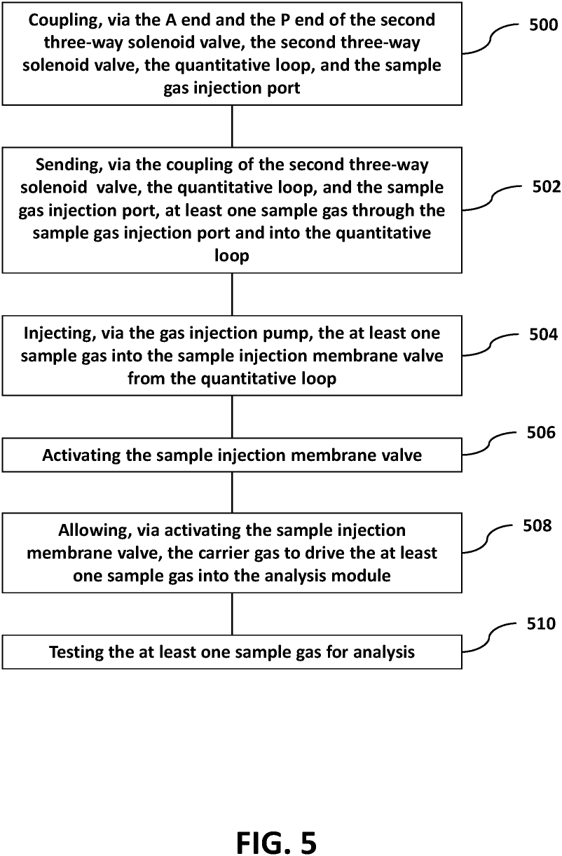

FIG. 5 illustrates a method of analyzing at least one sample gas. According to some embodiments, the method includes coupling, via the A end and the P end of the second three-way solenoid valve, the second three-way solenoid valve, the quantitative loop, and the sample gas injection port (at step 500). The method may include sending, via the coupling of the three-way solenoid valve, the quantitative loop, and the sample gas injection port, at least one sample gas through the sample gas injection port and into the quantitative loop (at step 502). In some embodiments, the method includes injection, via the gas injection pump, the at least one sample gas into the sample injection membrane valve from the quantitative loop (at step 504). According to some embodiments, the method includes activating the sample injection membrane valve (at step 506). The method may include allowing, via activating the sample injection membrane valve, the carrier gas to drive the at least one sample gas into the analysis module (at step 508). In some embodiments, the method includes testing the at least one sample gas for analysis.

### Interpretation

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1, and embodiments described in other sections may apply to and be combined with embodiments described within the “Topic 1” section.

To increase the clarity of various features, other features are not labeled in each figure.

The various features and processes described above may be used independently of one another or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, parallel, or some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless expressly stated otherwise, or otherwise understood within the context as used, is generally intended to convey that certain embodiments include, while other embodiments do not include, certain features, elements and/or steps. Thus, such conditional language is not generally intended to imply that features, elements, and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless expressly stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

While certain example embodiments have been described, these embodiments have been presented by way of example only and are not intended to limit the scope of the inventions disclosed herein. Thus, nothing in the foregoing description implies that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

What is claimed is:

1. A gas chromatograph for underground mine use, comprising:
a fixing board;
a power connection socket detachably coupled to the fixing board;
at least one three-way solenoid valve detachably coupled to the fixing board;
a quantitative loop detachably coupled to the fixing board, the quantitative loop arranged and configured to detachably couple to at least one of the at least one three-way solenoid valves;
an analysis module detachably coupled to the fixing board;
a communication main board detachably coupled to the fixing board;
a data transmission device detachably coupled to the fixing board;
a sample injection membrane valve detachably coupled to the fixing board, the sample injection membrane valve arranged and configured to detachably couple to at least one of the at least one three-way solenoid valves; and
a gas injection pump detachably coupled to the fixing board.

2. The gas chromatograph for underground mine use of claim 1, wherein the analysis module, the sample injection membrane valve, the gas injection pump, the quantitative loop, the at least one three-way solenoid valves, the data transmission device, the communication main board, and the power connection socket are detachably coupled to the fixing board with clips and screws.

3. The gas chromatograph for underground mine use of claim 2, further comprising a housing, wherein the fixing board is detachably coupled to a fixed column at a bottom of the housing with screws.

4. The gas chromatograph for underground mine use of claim 1, wherein the data transmission device further comprises parallel RJ45 and RS485 transmission interfaces arranged and configured to couple to the communication main board.

5. The gas chromatograph for underground mine use of claim 1, further comprising:
a sample gas injection port arranged and configured to detachably couple to at least one of the at least one three-way solenoid valves;
a sample gas exhaust port arranged and configured to allow a sample gas to flow out of the sample gas injection port;
a standard gas injection port arranged and configured to detachably couple to at least one of the at least one three-way solenoid valves;
a gas injection pump exhaust port arranged and configured to allow a standard gas to flow out of the standard gas injection port;
an analysis module exhaust port arranged and configured to allow the sample gas and the standard gas to flow out of the analysis module; and
a carrier gas interface arranged and configured to detachably couple to the analysis module and drive the sample gas and the standard gas into the analysis module through the use of a carrier gas.

6. The gas chromatograph for underground mine use of claim 5, wherein the analysis module includes a capillary chromatographic column equipped with a heating module, and
a micro TCD detector equipped with a heating module.

7. The gas chromatograph for underground mine use of claim 5, further comprising a housing, wherein the carrier gas interface, the gas injection pump exhaust port, the sample gas injection port, the standard gas injection port, the analysis module exhaust port, and the sample gas exhaust port are detachably coupled to the housing.

8. The gas chromatograph for underground mine use of claim 7, wherein the carrier gas interface, the gas injection pump exhaust port, the sample gas injection port, the standard gas injection port, the analysis module exhaust port, and the sample gas exhaust port further comprise a fixed nut and a joint body, the joint body filled in with powder metallurgy of particle size 60 to 80 mesh.

9. The gas chromatograph for underground mine use of claim 5, wherein the at least one three-way solenoid valve further comprises a first three-way solenoid valve and a second three-way solenoid valve.

10. The gas chromatograph for underground mine use of claim 9, wherein the first three-way solenoid valve further comprises:
an A end coupled to the sample injection membrane valve;
a P end coupled to the standard gas injection port; and
an R end coupled to the second three-way solenoid valve.

11. The gas chromatograph for underground mine use of claim 9, wherein the second three-way solenoid valve further comprises:
an A end coupled to the quantitative loop;
a P end coupled to the sample injection gas port; and
an R end coupled to the first three-way solenoid valve.

12. The gas chromatograph for underground mine use of claim 5, wherein the sample injection membrane valve further comprises a four-way valve coupled to a suction end of the gas injection pump, the quantitative loop, the carrier gas interface, and the analysis module.

13. The gas chromatograph for underground mine use of claim 12, wherein a sample inlet end of the sample membrane valve is coupled to the suction end of the gas injection pump when the sample membrane valve is not activated.

14. The gas chromatograph for underground mine use of claim 12, wherein a carrier gas interface end of the sample membrane valve is coupled to the carrier gas interface, and an analysis module connecting end of the sample membrane valve is coupled to the analysis module when the sample membrane valve is activated.

15. A method of using a gas chromatograph, wherein the gas chromatograph comprises a first three-way solenoid valve including an A end and a P end, a second three-way solenoid valve including an A end and a P end, a standard gas injection port, a sample injection membrane valve, a gas injection pump, a standard gas, a carrier gas, at least one sample gas, an analysis module, a quantitative loop, and a sample gas injection port, the method comprising:
calibrating the gas chromatograph; and
analyzing the at least one sample gas.

16. The method of using a gas chromatograph of claim 15, wherein calibrating the gas chromatograph further comprises:
powering on the first three-way solenoid valve;
coupling, via the A end and the P end of the first three-way solenoid valve, the standard gas injection port, the sample injection membrane valve, and the gas injection pump;
injecting, via the gas injection pump, the standard gas into the sample injection membrane;
activating the sample injection membrane valve; and
allowing, via activating the sample injection membrane valve, at least one of the at least one sample gas into the analysis module.

17. The method of using a gas chromatograph of claim 15, wherein analyzing the at least one sample gas further comprises:
coupling, via the A end and the P end of the second three-way solenoid valve, the second three-way solenoid valve, the quantitative loop, and the sample gas injection port;
sending, via the coupling of the second three-way solenoid valve, the quantitative loop, and the sample gas injection port, at least one sample gas through the sample gas injection port and into the quantitative loop;
injecting, via the gas injection pump, the at least one sample gas into the sample injection membrane valve from the quantitative loop;
activating the sample injection membrane valve;
allowing, via activating the sample injection membrane valve, the carrier gas to drive the at least one sample gas into the analysis module; and
testing the at least one sample gas for analysis.
